(19) United States
(12) Patent Application Publication
(10) Pub. No.: US 2006/0160234 A1
(43) Pub. Date: Jul. 20, 2006

(54) Methods for assessing polypeptide array quality

(76) Inventors: Viorica Lopez-Avila, Sunnyvale, CA (US); Dan-Hui Yang, Sunnyvale, CA (US); Magdalena Bynum, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 11/039,604

(22) Filed: Jan. 18, 2005

(51) Int. Cl. G01N 33/00; G06F 19/00

(52) U.S. Cl. 436/86; 702/19

(57) ABSTRACT

The invention provides a method of evaluating a feature on a polypeptide array. In general, the method involves reading a polypeptide array under intrinsic fluorescence-detecting conditions to produce data and assessing the data to evaluate the the feature. The invention finds use in a variety of medical and research applications.

### BACKGROUND OF THE INVENTION

Straightforward and reliable methods for simultaneously analyzing several constituents of a complex sample are extremely desirable. For example, it is desirable to determine the relative amounts of several analytes, e.g., proteins, in blood and other bodily fluids, in medical diagnostics and other fields. One technology that has been successfully used to provide such methods employs an array of polypeptide capture agents, otherwise known as a “polypeptide array” (see, e.g., U.S. Pat. Nos. 6,372,483, 6,352,842, 6,346,416 and 6,242,266).

In this technology, a surface of a substrate is usually derivatized to provide sites that are polypeptide-binding and a plurality of solutions of polypeptide capture agents are deposited onto the derivatized substrate surface to form a series of “features”, i.e., discrete areas on the surface of the array substrate, each area containing a different polypeptide capture agent. After depositing the polypeptide capture agents onto the substrate, the substrate is typically processed (e.g., washed and blocked for example) and stored prior to use.

In use, a polypeptide array surface is contacted with a sample or labeled sample containing analytes (usually, but not necessarily other polypeptides) under conditions that promote specific, high-affinity binding of the analytes in the sample to one or more of the capture agents present on the array. The level of binding of one or more capture agents of the array to labeled analytes in the sample is then quantified. The analytes in the sample may be labeled with a detectable label such as a fluorescent tag, or the analytes in the sample are detected by labeled entities and each labeled entity has a specific binding to one analyte of interest. Quantification of the level of fluorescence associated with a bound capture agent represents a direct measurement of the level of binding. In turn, this measurement of binding represents an estimate of the abundance of a particular analyte in the sample. A variety of biological and/or chemical compounds may be used as detectable labels in the above-described arrays (See, e.g., Wetmur, J. Crit Rev Biochem and Mol Bio 26:227, 1991; Mansfield et al., Mol Cell Probes. 9:145-56, 1995; Kricka, Ann Clin Biochem. 39:114-29, 2002).

The quality of data obtained from an assay employing a polypeptide array is highly dependent on the quality of an array prior to and during its use (e.g., prior to its contact with a sample). In particular, the quality of data obtained from a polypeptide array assay is highly dependent on the integrity of capture agent features. For example, polypeptide arrays containing features with an unexpectedly low amount of capture agent and polypeptide arrays containing features having an unusual morphology, if employed in a binding assay, would likely produce reduced-quality data.

Such inferior features are common in polypeptide arrays and may be due to factors such as imperfections in the substrate, imperfections in any derivatization of the substrate surface, incorrectly formulated capture agent solution or incorrectly deposited capture agent solution, for example. Such factors may cause incomplete binding of polypeptide capture agents to the substrate, allowing capture agents to be separated from the substrate during array fabrication or use, or during array processing. To date however, although there is a great need for such methods, there are no straightforward and reliable methods for evaluating a feature of a polypeptide array prior to its use. The present invention meets this need, and others.

Literature of interest include: Ge et al (Nucl. Acids Res. 2000 28:e3) and Striebel et al (Proteomics 2004 4:1703-1711).

### SUMMARY OF THE INVENTION

The invention provides a method of evaluating a feature on a polypeptide array. In general, the method involves reading a polypeptide array under intrinsic fluorescence-detecting conditions to produce data and assessing the data to evaluate the feature. In certain embodiments, the method may involve detecting intrinsic fluorescence of tryptophan residues contained in the polypeptides of the feature. In other embodiments, the method may involve detecting intrinsic fluorescence from a fluorophore present in a protein (hemoglobin, for example) or detecting intrinsic fluorescence from a compound (e.g., an additive) present in the polypeptide solution. Intrinsic fluorescence may be detected by, e.g., measuring absolute fluorescence intensity or fluorescence lifetime. Polypeptide arrays may be evaluated using the above-method, and selected for future binding assays based on their evaluation. Programming for performing the subject methods is also provided. The invention finds use in a variety of medical and research applications, e.g., proteomics and diagnostics.

### BRIEF DESCRIPTION OF THE FIGURES

FIG. 1 shows absorbance and emission spectra of phenylalanine, tyrosine and tryptophan.

FIG. 2 is a graph showing results obtained from a prescan of slide 309, containing an antibody array.

FIG. 3 is a graph showing results obtained from a prescan of slide 115, containing an antigen array.

FIG. 4 is a graph showing the effects of moist nitrogen on an antigen array.

FIG. 5 is two panels showing the effects of blocking on caspase 8 elements.

FIG. 6 is two panels showing the effects of blocking the morphology of certain features.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, virally infected cells, recombinant cells, and cell components).

Components in a sample are termed “analytes” herein. In certain embodiments, the sample is a complex sample containing at least about 1, 2, 20, 50, 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analytes.

The term “analyte” is used herein to refer to a known or unknown component of a sample, which will specifically bind to a capture agent on a substrate surface if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, polynucleotides to be evaluated by binding with the other).

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” also includes post translational modified polypeptides or proteins. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 9, 10, 20, 30 or 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of double stranded or single-stranded DNA may employed. Capture agents usually “specifically bind” one or more analytes.

Accordingly, the term “capture agent” refers to a molecule or a multi-molecular complex which can specifically bind an analyte

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the binding constant of a capture agent and analyte is greater than 10⁶ M⁻¹, greater than 10⁷ M⁻¹, greater than 10⁸ M⁻¹, greater than 10⁹ M⁻¹, greater than 10¹⁰ M⁻¹, usually up to about 10¹² M⁻¹.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to proteins, e.g., antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a binding constant of greater than about 10⁶ M⁻¹ to bind to each other, but not with other capture agents or targets.

As used herein, “binding partners”and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations. In certain embodiments, the collections of capture agents employed herein are present on a surface of the same support, e.g., in the form of an array.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., an known analyte that is of interest, is used synonymously with the term “pre-determined analyte”.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” polypeptide fragments are well known in the art and encompassed by the term “antibodies”.

The term “mixture”, as used herein, refers to a combination of elements, e.g., capture agents or analytes, that are interspersed and not in any particular order. A mixture is homogeneous and not spatially separated into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of capture agents, as is commonly known in the art and described below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

By “remote location,” it is meant a location other than the location at which the array is present and binding occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

The term “array” encompasses the term “microarray” and refers to an ordered array of capture agents for binding to aqueous analytes and the like.

An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 100 cm², 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, usually bearing biopolymeric capture agents, e.g., polypeptides, nucleic acids, and the like.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm, or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated by depositing (e.g., by contact- or jet-based methods) either precursor units (such as nucleotide or amino acid monomers) or pre-synthesized capture agent.

An array is “addressable” when it has multiple regions of different moieties (e.g., different capture agent) such that a region (i.e., a “feature” or “spot” of the array) at a particular predetermined location (i.e., an “address”) on the array will detect a particular sequence. Array features are typically, but need not be, separated by intervening spaces.

An “array layout” refers to one or more characteristics of the features, such as feature positioning on the substrate, one or more feature dimensions, and an indication of a moiety at a given location.

The term “intrinsic fluorescence” is used herein to describe detectable light emitted from an array in response to a stimulus light at a lower (i.e., shorter) wavelength than the detectable light, prior to contact of the array with a sample. A measurement of intrinsic fluorescence includes the measurement of detectable light from any chemical moiety present on the array, including, but not limited to: Trp, Tyr and/or Phe residue-containing polypeptides, fluorophore-containing proteins (e.g., proteins containing certain fluorophores such as hemoglobin) and certain compounds (e.g., additives) present in a polypeptide solution deposited to the array substrate during array fabrication. Intrinsic fluorescence is a property of an array that has not been contacted with a sample.

A method of “evaluating a feature” is a direct evaluation of the shape, size or position of a feature or the amount of polypeptide capture agent present in that feature (including whether or not a polypeptide capture agent is present in a feature). Such feature evaluation methods are distinguished from methods in which binding of an analyte to a feature is evaluated because such binding evaluation methods provide no direct evaluation of the shape, size or position of a feature or the amount of polypeptide capture agent present in that feature. Accordingly, methods of “evaluating a feature” may be performed separately and independently from (but could be combined with) methods in which binding of an analyte to a feature is evaluated. In other words, the term “evaluating a feature” is intended to describe measuring a physical property of a feature (e.g., the shape, position, size, or amount of polypeptide in the feature), and not measuring a property of an analyte bound to the feature (e.g., the shape, position, size, or amount of an analyte bound to the feature).

“Intrinsic fluorescence-detecting conditions”, as described in greater detail below, are particular conditions in which intrinsic fluorescence can be detected. Such conditions typically include exposing a polypeptide array to light having a wavelength that excites intrinsic fluorescence-emitting molecules (e.g., amino acids, fluorophores, other compounds in a polypeptide solution deposited onto the array), and detecting light emitted from those molecules in response to the exciting light. Intrinsic fluorescence-detecting conditions include conditions suitable for detecting the light emitted by array features prior to their contact with the analytes of a sample.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION OF THE INVENTION

The invention provides a method of evaluating a feature on a polypeptide array. In general, the method involves reading a polypeptide array under intrinsic fluorescence-detecting conditions to produce data and assessing the data to evaluate the feature. In certain embodiments, the method may involve detecting intrinsic fluorescence of tryptophan residues, fluorophores, or other intrinsically fluorescent compounds contained in the feature. Intrinsic fluorescence may be detected by measuring absolute fluorescence intensity or fluorescence lifetime. Polypeptide arrays may be evaluated using the above-method, and selected for future binding assays based on their evaluation. Programming for performing the subject methods is also provided. The invention finds use in a variety of medical and research applications, e.g., proteomics and diagnostics.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods for evaluating a feature of a polypeptide array are described first, followed by a description of protocols in which the subject methods find use. Finally, kits and programming, for use in practicing the subject methods are described.

Methods for Evaluating a Feature of a Polypeptide Array

In general, the invention provides a straightforward and reliable method by which features of a polypeptide array may be evaluated. The method may be performed at any time prior to array use, e.g., during array fabrication or storage, for example. The method does not involve labeling the arrayed polypeptides, requires no extra reagents, and requires a minimum of manipulations. As such, the instant methods represent a significant contribution to the art.

In describing the subject methods, arrays of polypeptide capture agents suitable for use in the subject methods will be described first, followed by a description of how the features of those arrays may be evaluated.

Arrays of Polypeptide Capture Agents

The subject invention involves an array of polypeptide capture agents. As described above, such an array generally comprises a plurality of spatially addressable features (e.g., more than about 10, more than about 100, more than about 500, more than 1000, features, usually up to about 10,000 to 100,000 or more features), and these features contain polypeptide capture agents. In certain embodiments, a single species of polypeptide capture agent is present in each of the features, however, in other embodiments, a feature may contain a mixture of different polypeptide capture agents.,

Methods for making arrays of polypeptide capture agents are generally well known in the art. For example, polypeptides may be produced in bacterial, insect or mammalian cells using recombinant means (see, e.g. Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) or synthetically made using a synthesizer, isolated, deposited onto a suitable substrate (e.g., a silanized glass substrate) and linked thereto.

Capture agents may be selected based on their binding to pre-determined analytes in a sample. Accordingly, in the subject methods, the pre-determined analytes and the capture agents that bind those analytes are selected prior to starting the subject methods. In other embodiments, the capture agents are not pre-determined and their binding specificity may be unknown.

Capture agents may be chosen using any means possible. For example, sets of capture agents present on an array may bind to proteins of a particular signal transduction, developmental or biochemical pathway, proteins having similar biological functions, proteins of similar size or structure, or they may bind proteins that are known markers for a biological condition or disease. Capture agents may also be chosen at random, or on the availability of capture-agents, e.g., if a capture agent is available for purchase, for example. In some embodiments, a capture agent may be chosen purely because it is desirable to know whether a known or unknown binding partner for that capture agent is present in a sample. The binding partner for a capture agent does not have to be known for the capture agent to be present on an array for use in the subject methods.

In certain embodiments, a single capture agent will bind to a single analyte. Accordingly, a set, i.e., a plurality, of capture agents for analysis is chosen. In certain embodiments, each of these capture agents binds to a single species of binding partner. In other words, since an array of capture agents usually contains more than about 4, more than about 8, more than about 12, more than about 24, more than about 48, more than about 96, more than about 192, or more than about 384 or more features containing different capture agents, etc., a corresponding number of different analytes may be present or may be suspected of being present in the sample to be assessed. In certain embodiments, there are about 50-10,000 different capture agents on a subject array.

Further, since the capture agents are chosen using any means possible, there is no requirement that any or all of the analytes for those capture agents are present in a sample to be analyzed. In fact, since the subject methods may be used to determine the presence or absence of an analyte in a sample, as well as the level of an analyte in a sample, only a fraction or none of the analytes may be present in a sample to be analyzed.

In particular embodiments of the invention, the capture agents employed are monoclonal antibodies, although any molecule that can specifically bind other moieties, e.g., other types of polypeptide, such as members of known binding partner pairs, other antibodies such as phage display polypeptides, and peptides or the like may be used. Monoclonal antibodies that specifically bind to analytes are well known in the art and may be made using conventional technologies (see, e.g., Harlow and Lane, Antibodies: A Laboratory Manual Cold Spring Harbor Laboratory, Cold Spring Harbor, N.Y. (1989)). Monoclonal antibodies that specifically bind to known analytes may also be purchased from a number of antibody suppliers such as Santa Cruz Biotechnology, Santa Cruz, Calif. and Epitomics, Inc., Burlingame, Calif. Peptides may be made synthetically by using standard chemical synthesis. For example, solid phase peptide synthesis method of Merrifield et al. (J. Am. Chem. Soc. 1964 85:2149) may be employed.

Polypeptide capture agents may be attached to a substrate surface using any of a wide variety of different compounds, including but not limited to poly-L-lysine (PLL), 3-glycidoxypropyltrimethoxysilane (GPS), DAB-AM-poly(propyleminime hexadecaamine) dendrimer (DAB) and 3-aminopropyltrimethoxysilane (APS). Attachment chemistries employing an amine-reactive group linked to the surface of a substrate via silane are of particular interest. Alternatively, polypeptide capture agents can be attached to the substrate surface by non-covalent interactions.

In general, a polypeptide array is fabricated by: a) depositing a polypeptide solution onto the surface of a polypeptide-reactive substrate (e.g., a silanized substrate), b) separating any unbound polypeptide from the array (e.g., by washing), and c) blocking any unreacted polypeptide-reactive groups or blocking the non-polypeptide binding surface on the substrate (e.g., by chemically reacting the unreacted groups or by binding them to a non-capture agent polypeptide such as BSA, casein, or the like). In certain embodiments, arrays may be shipped after steps b) or c) of this method. In use, a fabricated array is: a) contacted with a sample under analyte binding conditions, and b) read to provide data. Methods for making and using arrays of polypeptides are generally well known in the art (see e.g., U.S. Pat. Nos. 6,372,483, 6,352,842, 6,346,416 and 6,242,266 MacBeath and Schreiber, Science (2000) 289:1760-3) and do not need to be described here in any more detail.

Evaluation of a Feature

In general terms, the subject methods involve “evaluating a feature”, where, as discussed above, such methods directly evaluate (i.e., evaluate without the need for any labeling reagents, e.g., polypeptide labeling reagents) the shape, size or position of a feature, or the amount of capture agent present in a feature. The methods may be used as a quality control measure to evaluate whether a particular polypeptide array is of a suitable quality for use in binding assays, or whether a particular polypeptide array is of a suitable quality for shipping to a customer, for example.

The subject methods may be employed to evaluate any physical aspect of a feature, including the shape of the feature and whether or not any capture agent is present in the feature. In certain embodiments, the integrity of a feature may be evaluated by the subject methods.

The subject methods may be employed at any time during array fabrication or use. In particular embodiments, the subject methods are employed after deposition of the polypeptide samples onto the array and prior to contacting the array with a sample. In particular embodiments, therefore, the subject methods may be performed after polypeptide deposition onto the array and prior to blocking of the array, after blocking the array and prior to contacting the array with a sample, or at any time during storage of an array (which array may be blocked or unblocked).

In representative embodiments, the subject methods may be employed to evaluate polypeptide deposition onto a substrate to evaluate a particular polypeptide deposition condition (e.g., to evaluate a particular print buffer, substrate, deposition device, deposition temperature, derivatization chemistry or the like) or determine whether a printhead is clogged or becoming clogged, for example. In other representative embodiments, the subject methods may also be employed to evaluate binding of a polypeptide once it has been deposited onto an array. For example, the subject methods may be employed to determine whether the polypeptides of a feature have been effectively bound to the substrate, or whether a feature has smeared during processing, for example. In a particular embodiment, the subject methods may be employed to determine whether a polypeptide has been consistently deposited to the same feature of different slides during fabrication of a batch of slides (e.g., to determine whether the quality of printing decreases during printing of a batch of slides, for example).

The instant methods generally involve: a) reading a polypeptide array under intrinsic fluorescence-detecting conditions to produce data, and b) assessing that data to evaluate a feature of the polypeptide array.

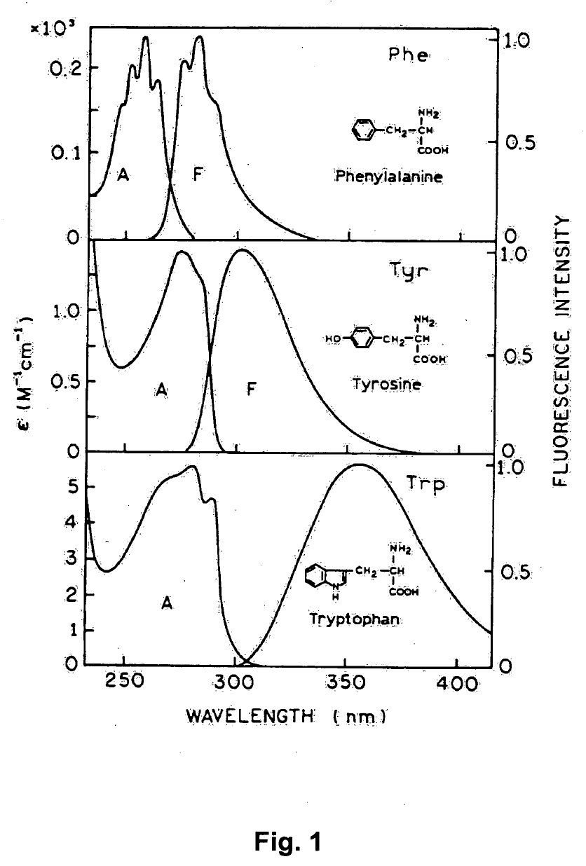

In one embodiment, intrinsic fluorescence is a well characterized energetic phenomenon associated with polypeptides containing amino acids that have an intrinsic fluorescence activity (i.e., a fluorescence activity that is produced by the amino acid itself, rather than an chemical modification to the amino acid). Such amino acids include aromatic amino acids, e.g., tryptophan, tyrosine, and phenylalanine. Because tryptophan fluoresces more strongly than tyrosine and phenylalanine and has highly distinct absorption/emission peaks, embodiments that include assessing intrinsic fluorescence of tryptophan residues are of particular interest. FIG. 1 illustrates the energies of absorbance (A) and fluorescence (F) plotted against wavelenth (nm), for each of phenylalanine (Phe), tyrosine (Tyr) and tryptophan (Trp). Table 1 below summarizes the fluorescence characteristics of Trp, Tyr and Phe. As can be seen from FIG. 1 and Table 1, the Trp, Tyr and Phe: maximally absorb at 280 nm, 274 nm and 257 nm, respectively; maximally fluoresce at 348 nm, 303 nm and 282 nm, respectively, and have a fluorescence lifetime (τ_{F}) of 2.6, 3.6 and 6.4 ns, respectively. Tryprophan residues are the easiest of the three residues to detect using intrinsic fluorescence, whereas phenylalanine is the least easy to detect.

TABLE 1

| | | AB- | | FLUO- | | | SENSI- |
|---|---|---|---|---|---|---|---|
| | | SORPTION | | RESCENCE | | | TIVITY |
| | Con- | λₘₐₓ | εₘₐₓ | λₘₐₓ | | τ_{F} | εₘₐₓ Φ_{F} |
| Substance | ditions | (nm) | ×10⁻³ | (nm) | Φ_{F} | (ns) | ×10⁻² |
| Tryptophan | H₂O, | 280 | 5.6 | 348 | 0.20 | 2.6 | 11.0 |
| | pH 7 | | | | | | |
| Tyrosine | H₂O, | 274 | 1.4 | 303 | 0.14 | 3.6 | 2.0 |
| | pH 7 | | | | | | |
| Phenylalanine | H₂O, | 257 | 0.2 | 282 | 0.04 | 6.4 | 0.08 |
| | pH 7 | | | | | | |

In other embodiment, intrinsic fluorescence may be emitted from a fluorophore associated with a protein, e.g. a naturally-occurring chromophore-containing protein such as hemoglobin. In still other embodiments, intrinsic fluorescence is emitted by compounds (e.g., additives) present in a polypeptide solution. Such a compound may be added to the polypeptide during the polypeptides' manufacturing process.

Accordingly, in many embodiments, the instant methods involve exposing a subject polypeptide array to light having a wavelength of 305 nm or below, or suitable based on instrumentation availability (e.g., in the range of about 250 nm to about 305 nm, e.g., about 270 to about 290 nm, about 295 to about 305 nm or about 280 nm) and detecting (e.g., by reading or scanning) light emitted from the array that has a wavelength of greater than about 305 nm. As can been from FIG. 1, light of 400 nm or greater may be emitted from intrinsically fluorescent amino acids, and, as such, the subject methods may involve detecting emitted light having a wavelength of greater than 305 to about 450 nm, or more. Methods that detect emitted light having a wavelength of about 320 nm to about 350 nm, e.g., about 350 nm may be employed in certain embodiments of the invention. For maximal sensitivity, excitation and emission wavelengths may be chosen to match the excitation and emission maxima of the polypeptides under investigation, or a fluorescent amino acid thereof (e.g., tryptophan). The polypeptides can be excited at a longer wavelength than indicated above and their emission can be detected at much longer wavelength due to the sensitivity of the instruments employed. For example, intrinsic fluorescence of polypeptides can be observed with 532 nm excitation and 550 nm emission. In one embodiment, the excitation light employed is of about 532 nm and the detected light (i.e., the emitted light) is of about 567 nm. In general terms, the emission wavelength employed is longer than the excitation wavelength employed.

Data representing the detected light may be recorded in any convenient form such as but not limited to a file containing pixel intensity values or an image of the array. As would be recognized by one of skill in the art, the instant methods may be implemented by measuring either or both absolute fluorescence or fluorescence lifetime of features of a subject array: both methodologies result in an accurate assessment of the level of intrinsic fluorescence of a feature.

The data may be assessed to evaluate a feature by a variety of means. For example, if the data is an image of an array, the data may be assessed by visually inspecting the image. A visual inspection of the image would reveal if a feature had an abnormal shape, size or position, or if a feature had an abnormal amount (e.g., an abnormally low amount or was not present) of polypeptide capture agent associated therewith, as compared to that of nearby features. For example, a visual inspection of such an image would indicate if a feature had smeared across the surface of the array (indicated by a feature having a comet-like shape rather than a circle-like shape, for example), or if the feature contained a reduced amount of capture agent (indicated by a feature that exhibits reduced fluorescence intensity or no detectable fluorescence, as compared to nearby features, for example). In other embodiments, the intrinsic fluorescence data may be processed in a similar manner to data obtained from binding assays (e.g., via “feature extraction”) to produce values of intrinsic fluorescence for each feature, and assessed by comparing those values to reference values. The reference values could be values of intrinsic fluorescence expected for those features based on a theoretical prediction (obtained by estimating the level of intrinsic fluorescence of a feature by the number of tryptophan residues in the polypeptide present in the features, for example) or an experimentally-determined reference value (obtained from features known to contain the same polypeptide present in the same or a different array). Any of the above methods may also be performed by computer software.

In certain embodiments, intrinsic fluorescence emissions of an array may be read by the same array scanner employed to read binding of analytes to the array. Accordingly, intrinsic fluorescence emissions may be read in the “green” and/or “red” channels presently employed in an array reader (such as, e.g., an Agilent Microarray Scanner). Accordingly, in certain embodiments, intrinsic fluorescence may be detected by exciting the polypeptides of a feature using light of about 532 nm and reading emitted light having a wavelength of 550 to 610 nm, or by exciting the polypeptides of a feature using light of about 633 nm and reading emitted light having a wavelength of 650-750 nm, for example.

As mentioned above, intrinsic fluorescence of the instant polypeptide capture agent features may be read at any time during the lifetime of an array (i.e., any time after depositing polypeptides onto the array and before disposal of the array). In certain embodiments, intrinsic fluorescence is evaluated prior to contacting the array with a sample. In these embodiments, intrinsic fluorescence of an array may be evaluated: after depositing polypeptide capture agents onto the surface of the array and before separating unbound capture agents from the array; after separating unbound capture agents from the array and before blocking unreacted reactive groups or blocking the surface area without binding to the polypeptides of the substrate; or after blocking of unreactive reactive groups on the surface of the array and before contacting the array with a sample. In alternative embodiments, intrinsic fluorescence may be evaluated after contacting the array with a sample, typically after the array had been contacted with the sample and any unbound analytes have been separated from the array by washing. A feature of a single polypeptide array may be evaluated more than once during its fabrication and subsequent use.

Computer-Related Embodiments

The invention also provides a variety of computer-related embodiments. Specifically, the array-evaluating methods described above may be performed using computer-readable instructions, i.e., programming. Accordingly, the invention provides computer programming for obtaining intrinsic fluorescence data from a polypeptide or polynucleotide array, and, in certain embodiments, assessing that data to evaluate a feature of a polypeptide or a polynucleotide array.

In certain embodiments, the methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “processor” references any hardware and/or software combination which will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In certain embodiments, the processor will be operable linkage, i.e., part of or networked to, the aforementioned workstation, and capable of directing its activities.

Utility

The subject methods may be generally employed as part of a procedure for checking the quality of a polypeptide or a polynucleotide array before or after its use. In certain embodiments, the instant methods may be employed to determine whether a polypeptide array is of sufficient quality for use in binding assays. In certain exemplary embodiments, an array may be assigned a score (e.g., a number or letter) indicating the quality of the array, and only arrays indicated by a particular score may be above the threshold and therefore of a sufficient quality for future use. Such quality scores, quality thresholds, and the like are well within the skill of one of ordinary skill in the art to obtain. In certain instances one of skill in the art may learn how to obtain such quality scores and quality thresholds by experience. For example, a skilled person may assess one or more sets of slides using the instant methods, and arbitrarily decide (e.g., judge) which slides are of sufficient quality for future use. In particular embodiments, an array is of sufficient quality for use in binding assays if it has a quality that is above an arbitrarily-assigned threshold quality. For example, an array that does not contain abnormally shaped features (e.g., comet-like or smeared features) or an array that does not contain features having abnormal capture agent content (e.g., features containing at least 50%, at least 70% at least 90% or at least 95% of the expected content of capture agent) may be of a sufficient quality for use in binding assays.

The methods may be employed to evaluate the reproducibility of array fabrication across a batch of arrays, or across different batches of array. In one exemplary embodiment, a plurality of polypeptide solutions are deposited on an substrate to make an array of features, and those features are evaluated by the instant methods. The results of this evaluation may be used to determine if a printer has “fired” correctly to deposit polypeptide solution onto the surface of the substrate. The instant methods may therefore be used to detect problems with an array printer (e.g., a blocked or clogged print head, for example).

In an embodiment of particular interest, certain polypeptide compositions may be known or may become known to emit less intrinsic fluorescence than other polypeptide compositions, or no detectable intrinsic fluorescence. For example, certain polypeptide compositions may contain a polypeptide that contains no intrinsically fluorescent amino acids. In this embodiment, features containing such a polypeptide composition may be indicated as being a low or no fluorescence feature. This indication may be incorporated into the methods described above to provide a means for recognizing features that are expected to exhibit low or no detectable intrinsic fluorescence. In such embodiments, the instant array evaluation methods may be performed and features exhibiting low or no detectable fluorescence may be detected. Certain of those features may be expected to exhibit low or no detectable intrinsic fluorescence, and, accordingly, an array containing features exhibiting low or no detectable fluorescence may still be suitable for future use.

In certain embodiments, therefore, the invention provides a method of making a polypeptide array, comprising: depositing polypeptide features onto a surface of a substrate; and evaluating those features according to the above-described array-evaluation methods. Polypeptide arrays having a satisfactory quality may be used in binding assays (i.e., used for assessing binding of analytes in a sample to the polypeptides of the array), or shipped (e.g., packaged and sent) to a remote location (e.g., a customer or distribution center) for future use. In certain embodiments, if an array is shipped, it may be shipped with results of an evaluation according to the above (e.g., an image of the array, feature intrinsic fluorescence intensity values or an electronic copy of the same present in a computer-readable medium). The instant invention further provides a method comprising receiving an array evaluated by the above-described evaluation methods, and performing a binding assay on such an evaluated array. In particular embodiments, a subject array evaluation may be retrieved from remote memory via a communication module through a communication channel (such as a network, including the Internet). In this configuration a unique key on the array (e.g., a barcode) may be read prior to, during, or after contact with a sample, and that unique key facilitates the retrieval of an evaluation of that array from a remote location via, e.g., the internet.

The subject methods may also be employed in selecting a polypeptide array from a plurality (e.g., 2 or more, 4 or more, 10 or more, 20 or more or 100 or more) of polypeptide arrays. This method generally involves evaluating a plurality of polypeptide arrays by the above-described methods, and selecting a polypeptide array having an quality above a threshold quality (e.g., selecting a polypeptide array that does not contain abnormal feature shape or content). Such an array may be shipped to a remote location, and may be received from a remote location.

Once a polypeptide has been subjected to the instant quality evaluation methods, an array may be employed in a variety of diagnostic, drug discovery, and research applications that include, but are not limited to, diagnosis or monitoring of a disease or condition (where analytes that are markers for the disease or condition are assessed), discovery of drug targets (an analyte whose level is modulated in a disease or condition is a drug target), drug screening (where the effects of a drug are monitored by assessing the levels of analytes), protein fingerprinting (where the profile, i.e., the expression levels of analytes are assessed in a variety of diseases or artificial conditions and the profile provides a fingerprint for that disease or condition), determining drug susceptibility (where drug susceptibility is associated with a particular profile of analytes), discovery of new binding partners (where an analyte that binds to a capture agent has not been previously identified) and research (where it is desirable to know the relative concentrations of a number of analytes in a sample, or, conversely, the relative levels of an analyte in two or more samples).

In certain embodiments, a sample is contacted with an array of polypeptide capture agents that specifically bind to analytes or labeled analytes in the sample under conditions suitable to produce capture agent/analyte complexes. The analytes bound in the capture agent/analyte complexes are detected using their label or are detected by another labeled entity binding specifically with analyte of interest, and a value corresponding to the abundance of particular analytes in the sample may be provided. Using software that is already available and commonly used in microarray technology, the obtained data may be compared with data obtained from other assays.

As would be recognized by one of skill in the art, the polypeptide arrays evaluated using the subject methods may be employed in so called “dual-color” assays, in which two different samples are distinguishably labeled (e.g., with Cy3 and Cy5 or the like) and simultaneously contacted with an array to provide results that indicate the relative abundances of analytes in the two samples.

Results from reading binding to the elements of an array may be adjusted in view of the array evaluation described above. For example, binding data obtained from abnormal features may be ignored or flagged as unreliable during future data processing or comparisons.

Results from reading an array may be raw results (such as fluorescence intensity readings for each feature in one or two or more color channels) or may be processed results such as obtained by rejecting a reading for a feature which is below a predetermined threshold and/or forming conclusions based on the pattern read from the array (such as whether or not a particular analyte may have been present in the sample). The results of the reading (processed or not) may be forwarded (such as by communication) to a remote location if desired, and received there for further use (such as further processing). Stated otherwise, in certain variations, the subject methods may be performed in a location remote to scanning. The data may be transmitted to the remote location for further evaluation and/or use. Any convenient telecommunications means may be employed for transmitting the data, e.g., facsimile, modem, internet, etc.

### EXAMPLES

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

Each array used contained eight identical sub-arrays and was fabricated by depositing polypeptides onto a functionalized glass substrate using a non-contact inkjet deposition device. Unless otherwise indicated, the printed slides were stored at room temperature in a nitrogen atmosphere. The polypeptides were deposited at two concentrations, 250 μg/mL and 500 μg/mL, and each polypeptide at each concentration was deposited in quadruplicate.

Scanning of the arrays was performed using a fluorescence scanning device (Agilent Technologies) using excitation light at a wavelength of 532 nm and detecting emitted light of a wavelength of 567 nm. Images were digitized as tiff files and the features were quantified using the Agilent Feature Extraction software. Data were exported to an ACCESS™ database for further processing and data visualization was performed in SPOTFIRE™.

### Example 1

### Intrinsic Fluorescence of Antibody Arrays

Several antibody arrays were fabricated. The antibodies on the antibody arrays specifically bind the following proteins: fractalkine, RANTES, IL-18, MCP-1, MCP-2, MCP-3, Eotaxin, IL-8, soluble E selectin, IGFBP-4, IL-6, CRP, CD40-L, TNF-alpha, interferon-gamma, TIMP-2, IL1-alpha, MMP-2, TIMP-1, SDF-1, osteopontin, MIP-1, IGF-1, IL-1 sRI, IL-7, IL-12 p40, IL-12 p70, leptin, ICAM-1, VCAM-1, P-selectin, IL-6sR, MMP-2/TIMP-2 complex, MMP-9, IL-1 beta, MMP-10, IGFBP-3, IGFBP-6, IL-5, and TNFRSF11B. Four polypeptides, including MMP-2, IL-7, P-Selectin and MMP-10, had two different capture antibodies deposited at different locations on the array. These capture antibodies and they were printed to verify array performance.

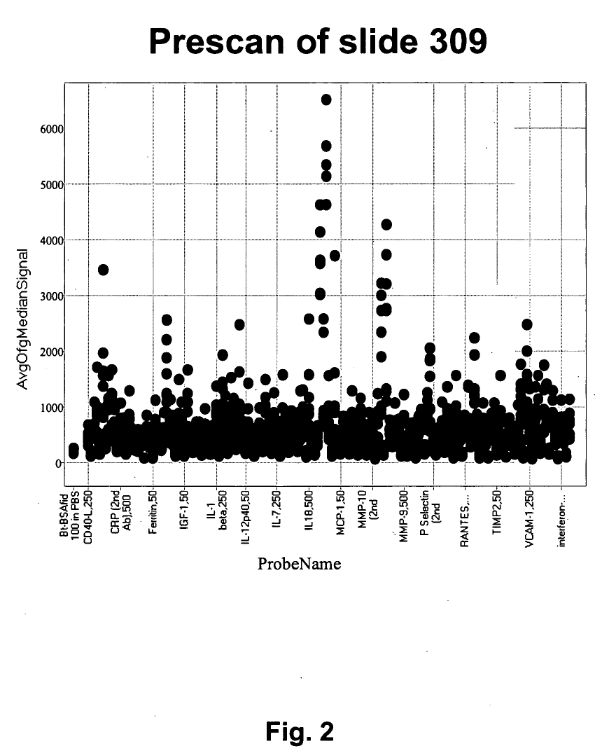

FIG. 2 shows data obtained from a prescan (i.e., a scan of a slide after fabrication and prior to blocking) of slide 309 (containing an antibody array) at 100% PMT. The vast majority of features gave rise to a signal of between 200 and 1500 as compared to a background signal (i.e., the signal obtained from areas between the features of the array) of about 30 and a signal of 35-50 for feature containing only phosphate buffer. The signal for the following probes is above 2000: CEA, human IgG, IL-12 p70, Il-18, laminin, leptin, MMP-10, rabbit IgG, and thyroglobulin. Intrinsic fluorescence is greater on slide 309 than for slide 110, a slide printed earlier than slide 309. The sub-arrays of slide 309 were printed reproducibly.

Slides 536, 636, 836 were printed later in the same day as slide 309 and slide 937 was printed two days later, after the printheads of the array printer had been cleaned. The overall intrinsic fluorescence of the features of slides 536, 636 and 836 was significantly greater than that of the features of slide 309 and the overall intrinsic fluorescence of the features of slide 937 is reduced as compared to that of slides 536, 636 and 836. The overall intrinsic fluorescence of the features of slide 937 was similar to that of slide 309, indicating that the printheads were becoming dirty or clogged during printing of slides 536, 636, 836, and that cleaning of the printheads of the array printer had restored its ability to print consistently.

The volumes used during printing with the ink jet printhead are small (in the few microliters range). Evaluating intrinsic fluorescence of the features produced by the printhead was straightforward compared to evaluating the concentration of each polypeptide solution during its deposition onto the array.

Slides 536, 636, and 836 were part of a much larger set of slides that were printed sequentially using the same antibody solutions. These solutions, which were loaded into the various reservoirs of a printhead, were apparently evaporating during the printing of the slides due to the heat released during the firing of the nozzle of the printhead. The evaporation was thought to have caused the increase in intrinsic fluorescence observed with slides 536, 636, and 836. Slide 936, which was printed two days later, was printed with the original solution in the source plate. The features of slide 936 produced reduced fluorescent signal across all features, which was comparable to that seen on the slides printed before slides 536, 636, and 836.

Additional experiments were performed to determine the number of slides that can be consistently printed during the course of one day. These experiments involved loading a print head with antibody solution, printing a set of arrays, and determining when print quality starts to decrease.

### Example 2

### Intrinsic Fluorescence of Antigen Arrays

Several antigen arrays were fabricated. The antigens on the antigen arrays were the following isolated proteins: bovine serum albumin, carbonic anhydrase, myoglobin, beta-lactoglobulin, alpha-amylase, beta-casein, actin, cytochrome C phosphorylase B, ovalbumin, phosphomannose isomerase, alkaline phosphatase, apotransferrin, glyceraldehyde 3-phosphate dehydrogenase, alpha-lactalbumin, catalase, enolase, alcohol dehydrogenase, glucose oxidase, peroxidase, alpha-chymotrypsinogen A, lysozyme, human IgG, cytochrome C, caspase 7, caspase 8, cathepsin D, gelsolin, cathepsin L, apolipoprotein E, thyroglobulin, src, trypsin, CAP1 -GST, FBP21-GST, alpha-fetoprotein, PSA, PSA alpha macroglobulin complex, guanylate kinase, and ferritin. In general, the intrinsic fluorescence signal strength of a feature was proportional to the number of tryptophans present in the polypeptide of that feature.

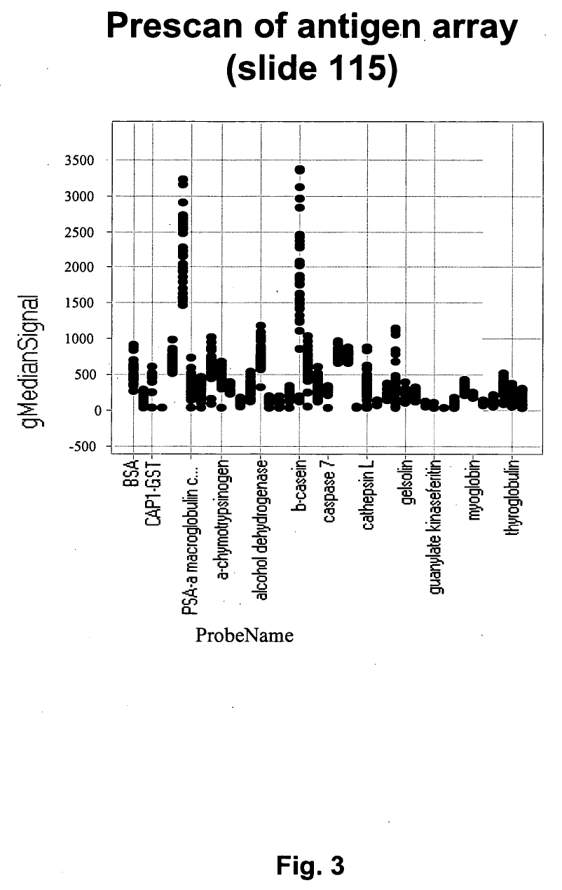

FIG. 3 show data obtained from a prescan of slide 115, an antigen array. Almost all signals obtained from these slides are between 50 and 1500. PSA and beta-casein exhibit signals above 2000 in both slides. A prescan of slide 116, a slide printed immediately after slide 115, produced near-identical results. Additional scans of other slides from the same print run indicate that antigen arrays are stable for at least several months if they are stored in a dessicator at 4° C. Between different slides, the print reproducibility has a coefficient of variation of about 20%.

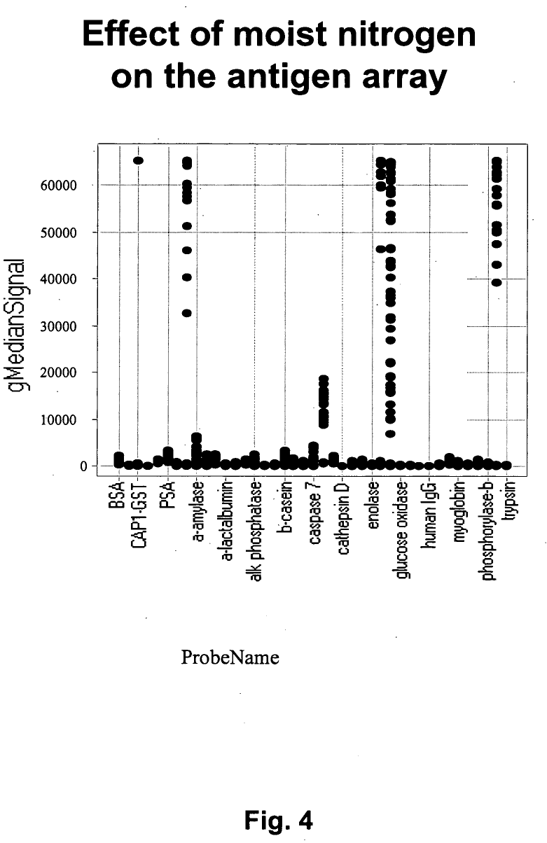

FIG. 4 shows data obtained from a prescan of slide 115 after it was kept in a dark chamber and purged with moist nitrogen for 24 hours. This treatment caused the intrinsic fluorescence of the CAP-1GST, Src, caspase 8, ferritin, gelsolin, and thyroglobulin features to significantly increase. We returned the moist nitrogen array to a dry nitrogen atmosphere, and the intrinsic fluorescence of CAP-1GST, Src, caspase 8, ferritin, gelsolin, and thyroglobulin features remained elevated. These results indicate that the integrity of an array may significantly depend on how the array is stored and that, in particular, polypeptide arrays should be stored in moist air. Thus, the stability of an array can be monitored using intrinsic fluorescence measurement.

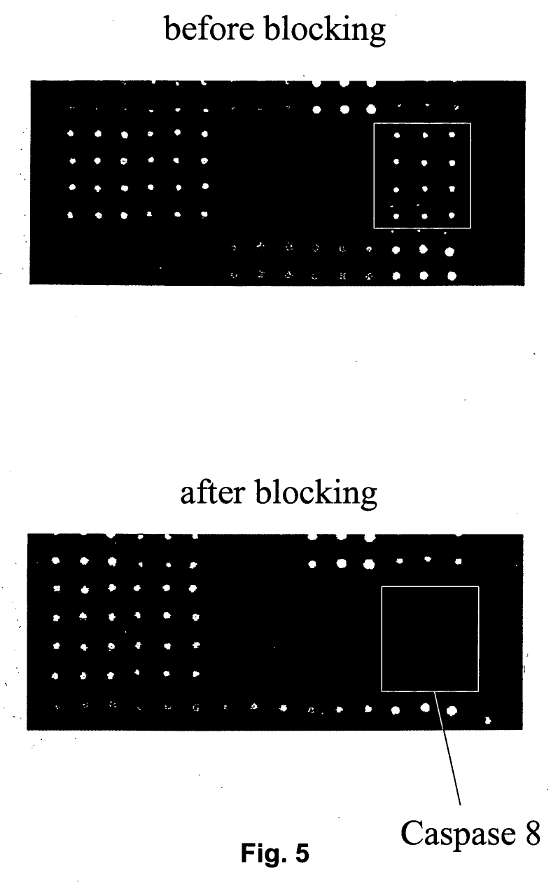

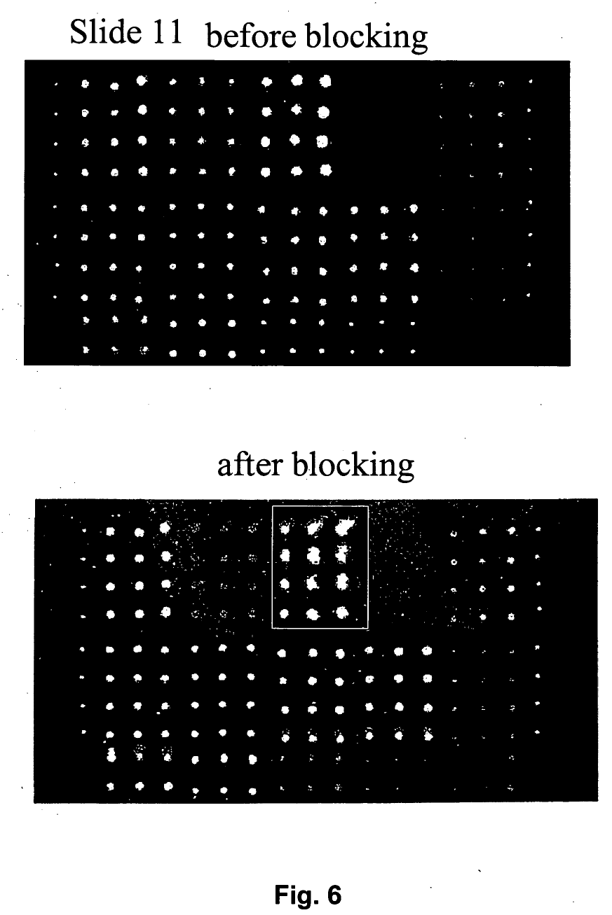

FIGS. 5 and 6 show scans of the antigen array (slide 111), before and after blocking with 1% BSA in phosphate buffer containing 5% sucrose (30 min blocking time). FIG. 5 shows that the signals of caspase 8 features are reduced by blocking the slide and FIG. 6 shows that the morphology of certain features is altered (e.g., certain features smear) by blocking the slide. These findings have been verified on slides 102 and 103, 105 and 106, 110 and 111, 107 and 143 at different times (slides 102, 105, 110 and 107 were prescanned and slides 103, 106, 111 and 143 were blocked and then scanned).

### Example 3

### Evaluating Printing of an Array by Assessing Intrinsic Fluorescence

Intrinsic fluorescence measurements were also employed to assess different print conditions (e.g., different buffers, different temperature, different protein concentration, different printers, etc.) as well as to determine whether any protein had been deposited (indicating possible mal-functioning of the printing device, e.g., clogging of a print-head).

Tryptophan solution and phosphate buffer were spotted onto a substrate and scanned using the green channel of an Agilent microarray scanner. Tryptophan features fluoresced brightly whereas phosphate buffer-containing features did not significantly fluoresce.

The above results and discussion demonstrate a straightforward and reliable new method for evaluating the quality of a polypeptide array. Specifically, the new methods may be used to evaluate the integrity of a feature of such an array. The methods may be repeated on a single array at one or more stages during array fabrication (e.g., immediately after printing, immediately after blocking, immediately after washing, or after contact with a sample, for example), and the data obtained at different stages of array fabrication may be compared to each other to identify a stage of array fabrication in which an array feature loses integrity. Such methods allow the selection of a polypeptide array that is of satisfactory quality for a particular binding experiment, before performing the experiment. Further, the methods provide straightforward means to optimize printing strategies. Accordingly, as such, the subject methods represent a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of evaluating a feature of a polypeptide array, comprising:
reading said polypeptide array under intrinsic fluorescence-detecting conditions to produce data; and
assessing said data to evaluate said feature of said polypeptide array.

2. The method of claim 1, wherein said polypeptide array is read prior to contacting said polypeptide array with a sample.

3. The method of claim 1, wherein said assessing includes evaluating a shape, size, position or presence of a polypeptide feature on said polypeptide array.

4. The method of claim 3, wherein said polypeptide array is read after blocking said polypeptide array but prior to contacting said array with said sample.

5. The method of claim 1, wherein said reading detects intrinsic fluorescence of tryptophan residues in polypeptides of said features.

6. The method of claim 1, wherein said reading detects intrinsic fluorescence of a fluorophore of a fluorophore-containing polypeptide of said features.

7. The method of claim 1, wherein said reading detects intrinsic fluorescence at a feature, wherein said intrinsic fluorescence is produced by compound present in a polypeptide solution prior to deposition of said polypeptide solution onto said feature.

8. The method of claim 1, wherein said reading includes exposing said polypeptide array to light having a wavelength of about 220-700 nm, and reading light emissions having a longer wavelength of about 240-720 nm.

9. The method of claim 1, wherein said reading includes measuring absolute fluorescence of said polypeptide array.

10. The method of claim 1, reading said reading includes measuring fluorescence lifetime.

11. The method of claim 1, wherein said data is an image of said array.

12. The method of claim 11, wherein said assessing includes visually inspecting said image of said array.

13. The method of claim 1, wherein said assessing involves comparing said data to reference intrinsic fluorescence values.

14. The method of claim 1, wherein said assessing involves comparing said data to theoretically-calculated intrinsic fluorescence values.

15. A method of making a polypeptide array, comprising:
depositing polypeptide features onto a surface of a substrate; and
evaluating said features according to the method of claim 1.

16. The method of claim 15, wherein said features are evaluated prior to blocking said array.

17. The method of claim 15, wherein said features are evaluated after blocking said array.

18. The method of claim 15, further comprising shipping said array to a remote location with said data.

19. A method of selecting a polypeptide array from a plurality of polypeptide arrays, comprising:
evaluating integrity of a plurality of polypeptide arrays by the method of claim 1; and
selecting a polypeptide array having an integrity above a threshold integrity.

20. The method of claim 19, wherein said polypeptide array having an integrity above a threshold integrity comprises a polypeptide array that does not have abnormally-shaped features.

21. The method of claim 19, wherein said polypeptide array having an integrity above a threshold integrity comprises a polypeptide array that does not have features containing an abnormally low amount of polypeptide.

22. The method of claim 19, further comprising shipping said polypeptide array to a remote location.

23. A method of detecting the presence of an analyte in a sample, said method comprising:
(a) contacting a sample with an array made by the method of claim 15;
(b) detecting any binding complexes on the surface of the said array to obtain binding complex data; and
(c) determining the presence of said analyte in said sample using said binding complex data.

24. A method comprising transmitting data obtained from a method of claim 23 from a first location to a remote location.

25. A method comprising receiving data representing a result of a reading obtained by the method of claim 23.

26. A method of evaluating reproducibility of a plurality of arrays, comprising:
evaluating a feature of said plurality of arrays using the method of claim 1, and
comparing an evaluation for one array of the plurality of arrays to that of a different array of the plurality of arrays.

27. A computer readable medium comprising programming for assessing data produced by reading a polypeptide array under intrinsic fluorescence-detecting conditions and evaluating said polypeptide array.
